(19) United States
(12) Patent Application Publication
(10) Pub. No.: US 2026/0102620 A1
(43) Pub. Date: Apr. 16, 2026

(54) HIGH FREQUENCY CURRENT DETECTION AND MODULATION IN FERRITIN-RICH TISSUE

(71) Applicant: Christopher J. Rourk, Dallas, TX (US)

(72) Inventor: Christopher J. Rourk, Dallas, TX (US)

(21) Appl. No.: 19/357,443

(22) Filed: Oct. 14, 2025

Related U.S. Application Data
(60) Provisional application No. 63/706,844, filed on Oct. 14, 2024.
(60) Provisional application No. 63/802,149, filed on May 8, 2025.
(60) Provisional application No. 63/831,318, filed on Jun. 27, 2025.
(60) Provisional application No. 63/874,793, filed on Sep. 3, 2025.

(51) Int. Cl. A61N 1/36; A61N 2/00

(52) U.S. Cl. CPC A61N 1/36139; A61N 1/36157; A61N 2/006

(57) ABSTRACT

A system for characterizing high frequency response of neural tissue, comprising a sensor configured to generate a sensed signal from a brain, a frequency analyzer configured to receive the sensed signal and to perform a frequency analysis of the sensed signal and a stimulation device configured to identify a frequency component from the frequency analysis and to generate a stimulation signal at the frequency component.

### RELATED APPLICATIONS

This application claims benefit of and priority to U.S. Provisional patent application 63/706,844, filed Oct. 14, 2024; U.S. Provisional patent application 63/802,149, filed May 8, 2025; U.S. Provisional patent application 63/831,318, filed Jun. 27, 2025; and U.S. Provisional patent application 63/874,793, filed Sep. 3, 2025, each of which are hereby incorporated by reference for all purposes as if set forth herein in their entireties.

### TECHNICAL FIELD

The present disclosure relates generally to biophysics, and more specifically to high frequency current detection and modulation in ferritin-rich tissue.

### BACKGROUND OF THE INVENTION

Ferritin is presumed to be an iron storage protein with no other biophysical functions, but it may have bioelectric and biomagnetic properties that have not been identified or studied.

### SUMMARY OF THE INVENTION

A system for characterizing high frequency response of neural tissue is disclosed. The system includes a sensor configured to generate a sensed signal from a brain and a frequency analyzer configured to receive the sensed signal and to perform a frequency analysis of the sensed signal. A stimulation device is configured to identify a frequency component from the frequency analysis and to generate a stimulation signal at the frequency component.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE SEVERAL VIEWS OF THE DRAWINGS

Aspects of the disclosure can be better understood with reference to the following drawings. The components in the drawings may be to scale, but emphasis is placed upon clearly illustrating the principles of the present disclosure. Moreover, in the drawings, like reference numerals designate corresponding parts throughout the several views, and in which:

FIG. 1 is a diagram of a system for deep brain sensing and stimulation, in accordance with an example embodiment of the present disclosure;

FIG. 2 is a diagram of an algorithm for performing deep brain stimulation and sensing, in accordance with an example embodiment of the present disclosure;

FIG. 3 is a diagram of a voltage waveform, in accordance with an example of embodiment of the present disclosure;

FIG. 4 is a diagram of a current waveform, in accordance with an example of embodiment of the present disclosure;

FIG. 5 is a diagram of current measurements in two different types of tissue, in accordance with an example embodiment of the present disclosure;

FIG. 6 is a diagram of a frequency transform of the 2 current measurements, which generally shows that the faster decaying spike of tissue 1 has higher frequency components than the slower decaying spike of tissue 2;

FIG. 7 is a diagram of an electrode, in accordance with an example embodiment of the present disclosure;

FIG. 8 is test data from a live rat showing impedance of tissue with normal levels of ferritin and neuromelanin, in accordance with an example embodiment of the present disclosure;

FIG. 9 is test data from a live rat showing impedance of catecholaminergic neuron tissue response, in accordance with an example embodiment of the present disclosure; and

FIG. 10 is an algorithm of a test process for inserting a probe into catecholaminergic neuron tissue, in accordance with an example embodiment of the present disclosure;

FIG. 11 is a diagram of a system for detecting and modulating ferritin-rich tissues at biologically relevant frequencies;

FIGS. 12A through 12E are diagrams of time domain and frequency domain current measurements in live rat neural tissue, in accordance with an example embodiment of the present disclosure; and

FIG. 13 is a diagram of a potentiostat measurement in brain tissue, in accordance with an example embodiment of the present disclosure.

### DETAILED DESCRIPTION OF THE INVENTION

In the description that follows, like parts are marked throughout the specification and drawings with the same reference numerals. The drawing figures may be to scale and certain components can be shown in generalized or schematic form and identified by commercial designations in the interest of clarity and conciseness.

This application claims benefit of and priority to U.S. Provisional patent application 63/706,844, filed Oct. 14, 2024; U.S. Provisional patent application 63/802,149, filed May 8, 2025; U.S. Provisional patent application 63/831,318, filed Jun. 27, 2025; and U.S. Provisional patent application 63/874,793, filed Sep. 3, 2025, each of which are hereby incorporated by reference for all purposes as if set forth herein in their entireties.

Brain tissue can include so-called gray matter, which is typically represented by non-myelinated neurons, and so-called white matter, which is typically represented by myelinated neurons. Myelinated neurons can contain ferritin and can have electrical properties that are different from non-myelinated neurons. Non-myelinated neurons can also contain ferritin, and can be surrounded by glial cells such as microglia and astroglia, which can have elevated levels of ferritin. In addition, neurons can contain neuromelanin, a complex material that is often formed from the waste processing mechanisms of neurons, but which can also accumulate in neuromelanin organelles in catecholaminergic neurons. The chemical and electrical environment of catecholaminergic neurons can generate compounds with triplet state electrons from the metabolism of dopamine and noradrenaline. Triplet state electrons are high energy electrons that are potentially damaging and which need to be neutralized by cells to prevent damage. The present disclosure can be used to detect and modulate a signaling mechanism that uses triplet state electrons as part of action selection in catecholaminergic neurons, as well as signaling over ferritin structures in tissues and biological systems in general.

It has been determined that electron conduction in ferritin structures can be measured from an applied voltage at an electrode. This property and the presence of ferritin structures can be used advantageously to index the location of a probe as it is being inserted into brain tissue or other tissues, and to apply electrical current in a manner that has previously not been used. For example, a deep brain probe will pass through neurons, and the ability to detect the location of the probe relative to such ferritin structures can be used to index it. Likewise, once the probe has been located in predetermined tissues, it can be used to stimulate that tissue according to certain electrical parameters, such as to cause electrons to move over ferritin structures, for bulk stimulation of neural tissue or in other suitable manners. Magnetic field stimulation can also be used to cause electrical responses in ferritin structures, and can be used at selected frequencies to cause beneficial effects in tissues such as to prevent pain or promote or inhibit other neural signaling.

The present disclosure describes systems and algorithms that advantageously use the previously unrecognized properties of ferritin in neural tissues and other tissues to sense currents and voltages at probe locations and to apply currents and voltages to the probe. While neural tissue is described, the present disclosure can be used with ferritin structures that are present in other tissues, including macrophages that are found in many tissues, such as the spleen, the liver, the lungs, and other suitable tissues. Detection and stimulation of such structures may prove to be beneficial but has been prevented by the inability to detect such structures or even recognize that they are present. Thus, the systems and algorithms disclosed herein have wide ranging application for a variety of different tissues to treat previously unrecognized conditions.

FIG. 1 is a diagram of a system 100 for deep brain sensing and stimulation, in accordance with an example embodiment of the present disclosure. System 100 allows an electrode to be used to stimulate and sense electron transport over ferritin structures in brain tissue, to improve the ability to detect the location of the electrode, and to perform treatments associated with the probe location. System 100 includes electrode stimulation system 102, wave form analysis system 104, electrode movement system 106 and user interface system 108, each of which can be implemented in hardware or a suitable combination of hardware and software.

Electrode stimulation system 102 can be implemented as one or more algorithms operating in a voltage waveform generator controller that can be loaded into working memory of a processor of the voltage waveform generator that when implemented causes the voltage waveform generator to allow a user to select and apply user selectable voltage and current waveforms to an electrode that has been inserted into brain tissue or other suitable tissue. In one example embodiment, the wave form can be a traditional sine wave waveform, a square wave waveform, or other suitable waveforms. In another example environment, the waveform can be modified to test specific tissue characteristics, such as a current response to a change in voltage over time for specific limited periods of time to detect broadband frequency responses in the tissue for the purpose of characterizing the tissue impedance frequency response, voltage responses to currents or other suitable data. Likewise, other suitable processes and systems can also or alternatively be used.

Waveform analysis system 104 can be implemented as one or more algorithms operating in a voltage or current sensor that can be loaded into working memory of a processor of the voltage or current sensor that when implemented causes the voltage or current sensor to measure voltage applied to an electrode, voltages measured at an electrode, current applied to an electrode, current measured at an electrode or other suitable data. In one example embodiment, the electrode can have a plurality of active regions or contacts, such that a voltage and current be applied between two electrodes and other voltages and currents can be measured between two different electrodes. Likewise, voltages and currents can be applied and measured at a plurality of different regions, using a plurality of different electrodes, and other suitable analysis processes can also alternatively be used. In one example embodiment, waveform analysis system 104 can perform signal analyses on the applied and measured waveforms, such as a fast Fourier transform other frequency-based analyses on waveforms, to determine frequency components of the components of the waveforms or for other suitable purposes.

Electrode movement system 106 can be implemented as one or more algorithms operating in a stepper motor controller that can be loaded into working memory of a processor of the stepper motor controller that when implemented cause the stepper motor to receive data from user interface system 108 or other suitable systems and can determine whether to continue moving the electrode or to wait for user interaction. In one example embodiment, electrode movement system 106 can move a stepper motor by predetermined increments, such as one micron, and can wait for waveform analysis data before actuating the next step or steps. Likewise, other suitable movement processes can also alternatively be used.

User interface system 108 allows a user to review waveform analysis data, movement data and other suitable data and to decide whether to apply treatment stimulation through electrode stimulation system 102. In one example embodiment, user interface system 108 allows a user to receive an alert when an electrode has entered region that requires further analysis, can allow the user to view maps of impedance/voltage/current along an electrode insertion route versus expected impedances/voltages/currents, other suitable data depicting the physical parameters that have been measured along the path that the electrode has followed, and can allow a user to review other suitable information before deciding whether to continue moving the electrode, to stimulate region or to perform other suitable functions.

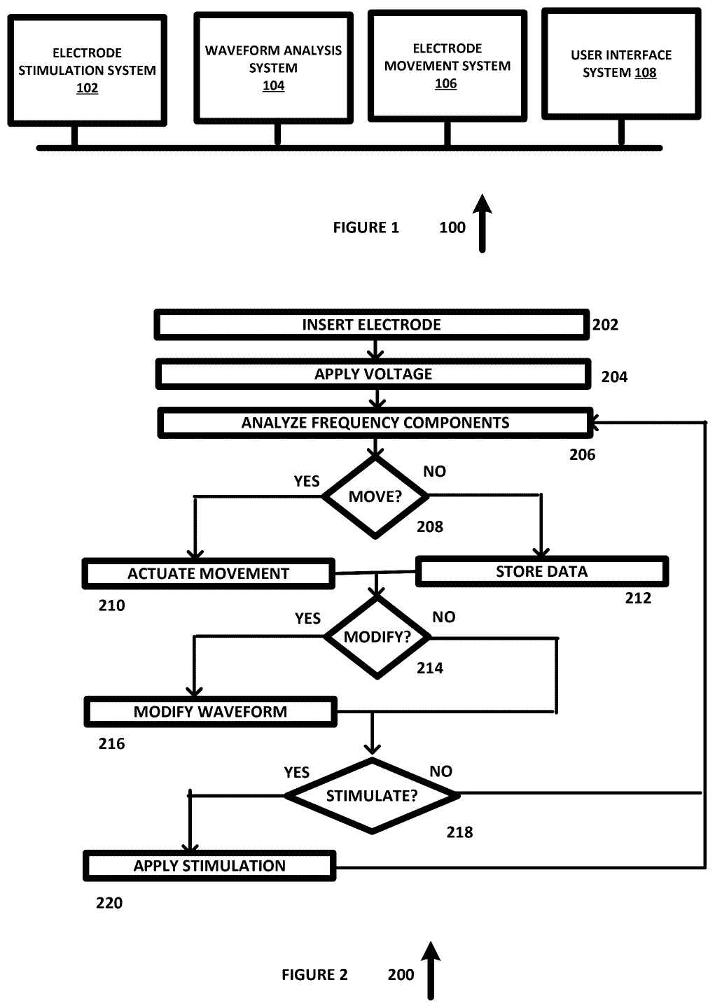

FIG. 2 is a diagram of an algorithm 200 for performing deep brain stimulation and sensing, in accordance with an example embodiment of the present disclosure. Algorithm 200 can be implemented in hardware or a suitable combination of hardware and software.

Algorithm 200 begins at 202, where an electrode is inserted into brain tissue or other suitable tissue. In one example embodiment, the electrode can be inserted through an incision made in the skull, the skin, or in other suitable manners. The algorithm then proceeds to 204.

At 204, a voltage is applied to the electrode. In one example embodiment, the voltage can be a predetermined waveform, a user-selected waveform, or other suitable processes can also alternatively be used. Prior to applying the voltage, the user can be prompted to determine whether to modify the voltage or if other suitable processes can be implemented. A current, magnetic field, electric field or other suitable stimulations can also or alternatively be applied, such as measure the voltage or current response to an applied magnetic or electric field.

At 206, frequency components from the applied voltage are measured. In one example embodiment, the frequency components can be current frequency components from the current that was measured from the applied voltage. In another example embodiment, the frequency components could be voltage or current frequencies measured at the electrode from the tissue or other electrodes, the characterize the tissue frequency response at high frequencies above normal neural firing frequencies, or other suitable data. The algorithm proceeds to 208.

At 208, is determined whether to move the electrode. For example, if the frequency components of the measured current in response to a step change in voltage have a magnitude that is above a predetermined magnitude value, such as 10 percent of the peak current or other suitable values, and if the bandwidth to a first minimum is narrower than a predetermined bandwidth value, such as 500 Hz, then the electrode can be moved, whereas if the frequency components having a magnitude that is above the predetermined magnitude value extend above the predetermined bandwidth value, then the probe can be stopped for further analysis. If the electrode is not moved the algorithm proceeds to 212 and the data that has been measured is stored. The algorithm then proceeds to 214. If it is determined to move the electrode, the algorithm proceeds to 210 and the movement controller is actuated, such as to actuate a stepper motor or in other suitable embodiments. The algorithm proceeds to 214.

At 214, it is determined whether to modify a wave form. In one example embodiment, a single electrode can be used to measure an applied voltage and to measured voltages, applied currents and measured currents, and other suitable data. Likewise, combinations of contacts on a multi-contact electrode, multiple electrodes or other suitable embodiments can also or alternatively be used. If the location of the electrode is determined to be proper, the applied waveforms can be modified to increase the frequency, the amplitude, the waveform can be modified, or other suitable processes can be implemented. If the determination is made not to modify the waveform, the algorithm proceeds to 218, otherwise the algorithm proceeds to 216 where the waveform is modified, and the algorithm proceeds to 218.

At 218, it is determined whether stimulation is required. In one example embodiment, a single electrode can be used to sense and stimulate brain tissue. In another example embodiment, multiple electrodes can be used, electrodes can be used for other suitable tissues, or other suitable processes can also or alternatively be used. If it is determined that stimulation is not needed at 218, the algorithm returns to 206. Otherwise, the algorithm proceeds to 220 where stimulation is applied, and the algorithm then proceeds to 206.

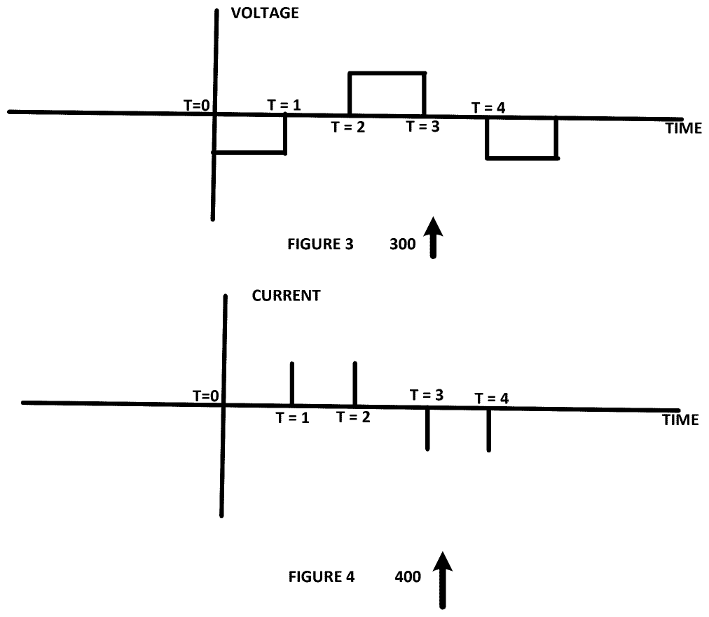

FIG. 3 is a diagram of a voltage waveform 300, in accordance with an example embodiment of the present disclosure. In this example, the applied voltage starts at T=0 and minus one volts or other suitable negative values can also or alternatively be used. The applied voltage is then increased in a rapid increase step operation at T=1 to 0 volts or other suitable values, where T can be any suitable period. The step operation will result in a current spike associated with a charging current of the membrane capacitance, but if additional high impedance current conduction paths are present, the high frequency current spike will contain broadband frequency components that will apply an electromotive force and result in high frequency current components. In addition, the stimulation will cause a response from the surrounding neurons, which can also generate current components that can be detected. After a suitable additional interval, the voltage can be increased at T=2 to +1 volts or other suitable values. After another suitable interval, the voltage can be decreased at T=3 k to zero or other suitable values. Likewise, at T=4, the voltage can be decreased to −1 or other negative values. In this manner, step changes can be applied to neural tissue or other suitable tissue, to determine the frequency characteristics of an applied current. Likewise, other suitable voltage or current waveforms can also or alternatively be used.

FIG. 4 is a diagram of current measurements, in accordance with an example embodiment of the present disclosure. At T=0, no current is measured, although an initial current can be measured when the initial voltage is applied, where suitable. At T=1 and T=2, positive current spikes are measured, and at T=3 and T=4, negative current spikes are measured. In this example embodiment, the spikes are represented as instantaneous spikes, but in practical applications, the spikes will be a high di/dt current pulse (e.g. greater than 10 microamperes increase in less than 50 microseconds with a subsequent drop to 90% of the maximum magnitude in less than 200 microseconds, or other suitable high dt/dt pulse parameters) of finite duration with a predetermined waveform, such as a triangular waveform with a predetermined rise and a function of capacitance, resistance, inductance and other circuit components between the two electrodes. The disruption of the electrical environment and properties of the neural tissue can also generate current components that can be measured. Likewise, if an expected current spike such as the one at T=3 is not measured, then the non-measurement can indicate that the probe is located adjacent to a predetermined tissue type, such as tissue containing ferritin or other suitable tissue structures. In addition, the absolute magnitude of the current reading can provide an indication of a change in tissue parameters, or other suitable data can also or alternatively be used.

If the current waveform is transformed from the time domain to the frequency domain, then the frequency components of the waveform can be identified and used to determine the location of the probe. For example, assuming a triangle waveform or other excitation voltage that can be represented as a fundamental frequency and harmonic components, the following frequency components that are greater than a threshold value can be measured as a function of probe location:

| | |
|---|---|
| Location | Frequencies |

| | | | |
|---|---|---|---|
| 1 | Fundamental | | |
| | and harmonics | | |
| 2 | Fundamental | | |
| | and harmonics | | |
| 3 | Fundamental | 2700 Hz | 3100 Hz |
| | and harmonics | | |
| 4 | Fundamental | | |
| | and harmonics | | |
| 5 | Fundamental | 4200 Hz | |
| | and harmonics | | |
| 6 | Fundamental | | |
| | and harmonics | | |
| 7 | Fundamental | 3800 Hz | 4700 Hz |
| | and harmonics | | |

As shown in this example, the frequency components at locations 1, 2, 4 and 6 are the same, which indicates that they have the same type of tissue structures. In contrast, the additional frequency components measured at locations 3, 5 and 7 indicate that a different tissue structure is adjacent to the probe. Thus, not only can the actual frequency components themselves identify physical parameters about the tissue, but the change in frequency components can also or alternatively be used to determine physical parameters of the tissue, such as layer structures. The total energy of frequency components below a threshold or within a predetermined range can also or alternatively be used to analyze the state of tissue.

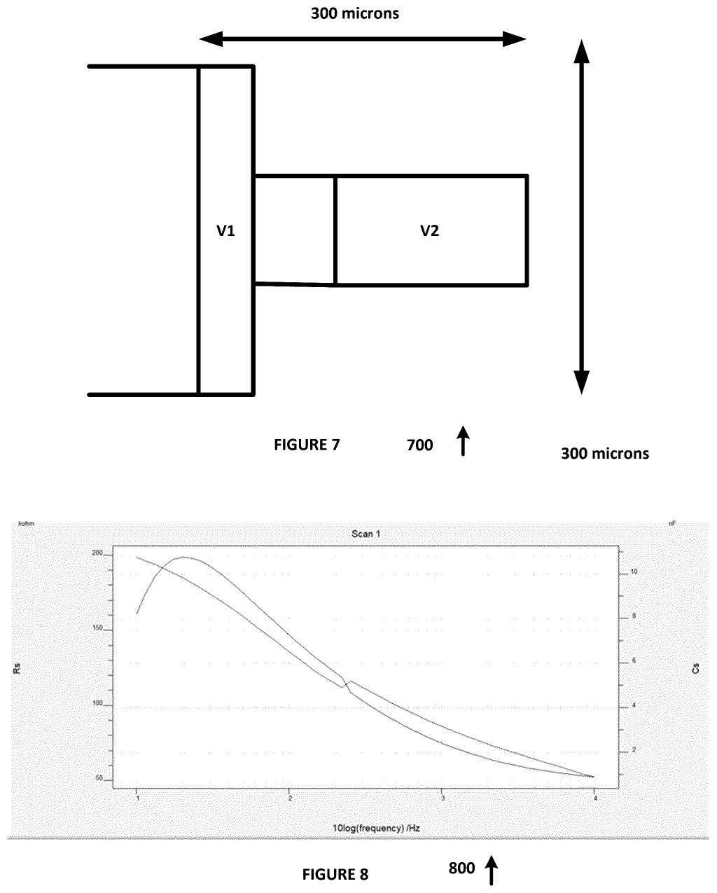

FIG. 5 is a diagram of current measurements 500 in two different types of tissue, in accordance with an example embodiment of the present disclosure. In tissue 1, the current response to a step change in voltage across an electrode, such as electrode 700 as shown in FIG. 7, can be a current spike. The step change in voltage charges tissue capacitances, and the current spike decays as a function of the resistive dissipation of the stored energy in the tissue. In tissue 2, the current spike decay is slower, indicating a slower dissipation of the stored energy.

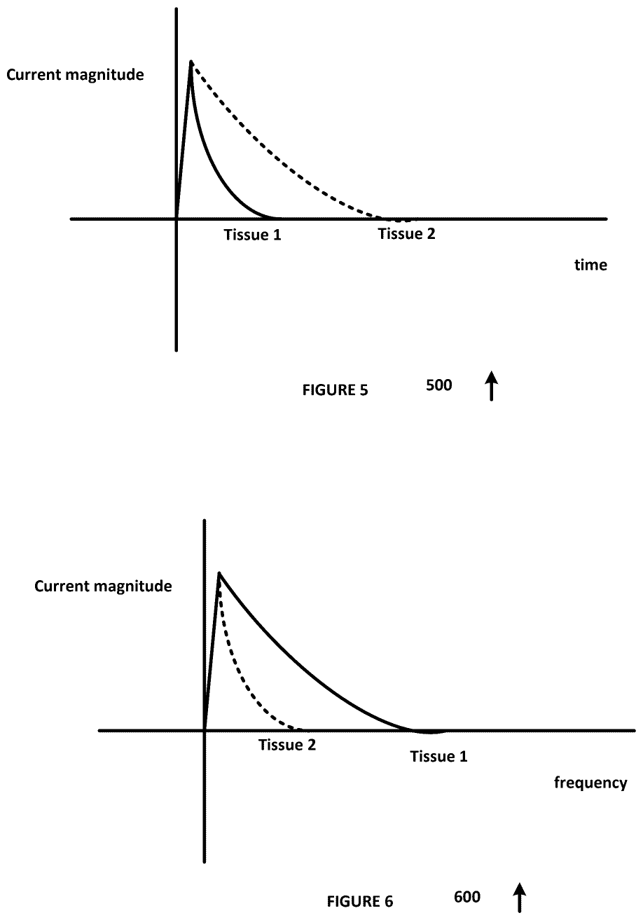

FIG. 6 is a diagram of a frequency transform of the 2 current measurements, which generally shows that the faster decaying spike of tissue 1 has higher frequency components than the slower decaying spike of tissue 2. These differences indicate that the material properties of tissue 1 are different from the material properties of tissue 2. For example, tissue 1 could be catecholaminergic neuron soma such as the substantia nigra pars compacta and the locus coeruleus, which have high concentrations of ferritin and neuromelanin that can act as a conducting medium, whereas tissue 2 may be ordinary neural tissue that has membrane capacitances but not sufficient levels of ferritin and neuromelanin to act as a conducting medium. Thus, detecting a change in the frequency response from tissue that has a smaller number of low magnitude high frequency current components to tissue that has a larger number of high magnitude, high frequency current components can be used to detect a transition from neural tissue outside of the group of catecholaminergic neuron soma into tissue that contains those soma, or from neural tissue that has high levels of ferritin and which is carrying electrical currents associated with those high levels of ferritin. In this manner, the neural tissue that can be monitored for the higher frequency signals that are representative of a neural signaling mechanism can be quickly identified and the neural signals over that signaling medium can be modulated in response to the detected signals.

FIG. 7 is a diagram of an electrode 700, in accordance with an example embodiment of the present disclosure. Electrode 700 is a coaxial electrode that has a first terminal formed by an outer concentric conductor that is identified by V1, and a second terminal formed by an inner central conductor that is identified by V2. The electrodes are separated by an insulator, and the concentric conductor V1 is covered by an insulator. The diameter of electrode 700 is 300 microns, and the distance between the base of V1 and the tip of V2 is 300 microns. Voltage waveform 300 can be applied to electrode 700 to generate current measurements 400, but other suitable electrode configurations can be used to apply other suitable voltage waveforms and to generate other suitable current measurements, in accordance with the disclosed embodiments, and in order to detect changes in tissue frequency response to identify a transition to catecholaminergic neural tissue from surrounding tissue. For example, many deep brain stimulation electrodes have multiple contact points and configurations, which can also or alternatively be used with suitable adaptations that will be apparent to one of ordinary skill in the art.

FIG. 8 is test data 800 from a live rat showing impedance of cortical tissue with normal levels of ferritin and neuromelanin, in accordance with an example embodiment of the present disclosure. The rat (and rats used for all rat test results reported herein) was anesthetized during the procedure and was handled in accordance with accepted principles for the ethical care and use of animals. Test data 800 was generated using an electrode having dimensional similarity to electrode 700 and an Ivium potentiostat available from Ivium Technologies of Eindhoven in the Netherlands, and shows that the electrical impedance spectroscopy of the rat brain tissue generally follows the pattern of decreasing in both resistance and capacitance as the frequency increases. It is noted that at about 250 Hz, both the resistance and the capacitance change, with the capacitance increasing and the resistance slightly decreasing, which is believed to be due to a device variation and not to represent an actual tissue impedance variation. The accuracy of a potentiostat device used in accordance with the present disclosure should be determined and suitable error margins can be used where needed to accommodate device tolerances.

In particular, the resistance slightly increases from ˜160 kohm to ˜200 kohm from 10 Hz to 20 Hz, and then decreases from 20 Hz to 10,000 Hz. The capacitance decreases from 10 nanofarads at 10 Hz to less than one nanofarad at 10,000 Hz. The measurements were taken at 50 intervals, starting at 10 Hz and increasing by 15% at each successive measurement. This electrical impedance behavior of the neural tissue was observed at all depths of the rat brain in cortical tissue and sub-cortical tissue outside of the basal ganglia for sets of measurements on different rats, although at some low impedances the resistance did not increase before decreasing, but instead decreased from a peak at 10 Hz.

FIG. 9 is test data 900 from a live rat showing impedance of catecholaminergic neuron tissue response, in accordance with an example embodiment of the present disclosure. The rat was anesthetized during the procedure and was handled in accordance with accepted principles for the ethical care and use of animals. Test data 900 was generated using an electrode having dimensional similarity to electrode 700 and an Ivium potentiostat available from Ivium Technologies of Eindhoven in the Netherlands, which as inserted into the substantia nigra pars compacta at a depth of 8.4 mm, and shows that the electrical impedance spectroscopy of the rat brain tissue generally follows the pattern of decreasing in both resistance and capacitance as the frequency increases from 10 to 500 Hz, similar to what was seen at all other depths. However, several notable differences are that the capacitance starts at ˜30 nanofarads at 10 Hz, instead of ˜10 nanofarads at 10 Hz. It is further noted that at about 1000 Hz, both the resistance and the capacitance rate of change starts to increase in a manner that is consistent over multiple measurements, unlike the single step change of the capacitance the resistance in test data 700, which is believed to be due to tissue impedance variation and not a device variation.

In particular, the resistance slightly increases from ˜70 kohm to ˜80 kohm from 1000 Hz to 10,000 Hz, and then decreases (readings above 10,000 Hz may be out of the accuracy range of the measuring equipment). The capacitance decreases from 30 nanofarads at 10 Hz to ˜10 nanofarads at 1000 Hz, and then experiences an excursion by increasing to ˜15 nanofarads at ˜3000 Hz before dropping off to ˜1 nanofarad at 10,000 Hz. The measurements were taken at 50 intervals, starting at 10 Hz and increasing by 15% at each successive measurement. This electrical impedance behavior of the neural tissue is believed to be indicative of ferritin and neuromelanin in the soma of the substantia nigra pars compacta neurons, and provides a mechanism whereby those neurons can be detected and modulated in accordance with the present disclosure.

Similar behavior was seen at other depths around 8.4 mm as shown below in Table 1, but with notable variations. As a preliminary matter, it is noted that the electrode was inserted to a maximum depth of 8.6 mm before being retracted, in order to mark the probe insertion path with dye, so the ferritin and neuromelanin tissue structures may have stuck to the probe. High levels of adhesion have been reported for those tissue structures using atomic force microscopy, see Rourk C J. Indication of quantum mechanical electron transport in human substantia nigra tissue from conductive atomic force microscopy analysis. Biosystems. 2019 May 1; 179:30-8. As such, the following capacitance measurements may have been due to tissue from 8.4 mm sticking to the probe, or may have been due to additional structures.

TABLE 1

| | Peak | Capacitance excursion |
|---|---|---|
| depth | capacitance | range, Hz |
| 7.6 mm | 18 nanofarads | ~1000 to ~4000 |
| 7.8 mm | 65 nanofarads | ~1000 to ~5000 |
| 8.2 mm | 23 nanofarads | ~1000 to ~4000 |

At 8.0 mm, the resistance and capacitance exhibited further unusual behavior, with the values shown below in Table 2.

TABLE 2

| freq./Hz | Rs/kohm | Cs/nanofarads |
|---|---|---|
| 10 | 123 | 30.17218 |
| 40.95 | 101 | 22.92545 |
| 109.9 | 82 | 17.7563 |
| 449.8 | 61 | 12.66656 |
| 517.9 | 50 | 12.41442 |
| 1207 | 54 | 12.57872 |
| 1390 | 54 | 13.18358 |
| 1600 | 54 | 14.11494 |
| 1842 | 54 | 15.61529 |
| 2121 | 54 | 18.06747 |
| 2442 | 54 | 22.47249 |
| 2812 | 54 | 31.10899 |
| 3237 | 55 | 53.54228 |
| 3728 | 56 | 202.643 |
| 4292 | 57 | −136.027 |
| 4942 | 58 | −65.1069 |
| 5690 | 59 | −67.6195 |
| 6551 | 60 | −2397.48 |
| 7543 | 61 | 33.01008 |
| 8685 | 62 | 10.05461 |
| 10000 | 63 | 4.720107 |

As can be seen, the capacitance was negative from 4292 Hz to 6551 Hz, with a peak negative value of −2.4 microfarads, significantly greater than the positive capacitance values at that depth. Such high negative capacitance values indicate that the ferritin and neuromelanin structures form a supercapacitor with high energy storage density, which may result from the capability of ferritin to effectively store electrons for hours. Negative capacitance is a property of ferroelectric materials, like ferritin in tissue. See Lynch K A, Ponomareva I. Negative capacitance regime in ferroelectrics demystified from nonequilibrium molecular dynamics. Physical Review B. 2020 Oct. 1; 102 (13):134101, which is hereby incorporated by reference for all purposes as if set forth herein in its entirety. However, while ferritin includes a core that consists of ferrihydrite, ferrihydrite precursors and related iron oxides, it is also able to effectively store electrons for hours. The negative capacitance measurements could also result from such stored electrons, which would be a function of a neural signaling mechanism that uses the ferritin and neuromelanin in the substantia nigra pars compacta neurons. These results demonstrate that the ferritin and neuromelanin in substantia nigra pars compacta tissue can exhibit negative nonlinear and location specific capacitance behavior at frequencies between 4000 and 7000 Hz when measured using a bipolar probe having dimensions similar to those of electrode 600. These electrical properties can be used to both sense signaling over the associated neural signaling media and to modulate those signals, providing two-way brain computer interface functionality. These electrical signals can modulate neural function in a manner that is different from the prior art, by causing release of stored iron from ferritin that contributes to calcium signaling, see Hidalgo, Cecilia, and Marco T. Núñez. “Calcium, iron and neuronal function.” IUBMB life 59.4-5 (2007): 280-285 and Rourk C. Comment on Albantakis et al. Computing the Integrated Information of a Quantum Mechanism. Entropy 2023, 25, 449. Entropy. 2023 Oct. 11; 25 (10):1436, which are hereby incorporated by reference for all purposes as if set forth herein in their entirety.

In addition, the distance between the probe location for these measurements and the adjacent measurements that provided no indication of negative capacitance was only 200 microns, which is a significant increase in probe location sensitivity compared to the prior art, which is generally only accurate to 1 mm. In addition, the prior art typically requires the use of additional diagnostic procedures to determine the location of the probe, such as computed tomography, x-ray or magnetic resonance imaging, whereas the present disclosure allows the location of the probe to be more accurately located without the need for such additional diagnostic techniques. Even if the probe is not ultimately located in the substantia nigra pars compacta or another catecholaminergic neuron group, it can be accurately indexed relative to a catecholaminergic neuron group. However, the unusual electrical properties of catecholaminergic neuron groups increase the potential for development of therapeutic uses of deep brain stimulation at lower power and with greater control over the therapeutic effects, because the frequency response of the specific tissue that is being stimulated can be measured and signals can be optimized for modulation of those tissues. It is noted that these electrical characteristics were not uniformly measured in catecholaminergic neurons tests and reported herein, and may have been a result of the specific placement of the probe, the level of anesthesia or type of anesthesia used for the tested rat (isoflurane), or other variations that may be difficult to replicate.

In addition, the present disclosure is not limited to deep brain stimulation probes, but can also or alternatively be used in conjunction with implantable probes, such as those disclosed in U.S. Pat. No. 8,989,867, which is hereby incorporated by reference for all purposes as if set forth herein in its entirety or other suitable implantable probes that can receive radio frequency signals. For example, instead of using a probe like electrode 700, a plurality of nanoscale radio frequency controllable probes could be injected into a catecholaminergic neuron group, and an adjacent radio frequency controller could be implanted, to avoid issues related to the movement of deep brain stimulation probes. Implanted nanoprobes will have a more stable placement and will not move with movement of the user, as is commonly observed with deep brain stimulation probes. The location of the injected probes can be determined using conventional radio frequency location technology, and the probes can be activated as needed for therapeutic or brain computer interface functionality. An ad hoc networking protocol can be used to form a network of wireless probes for improved communication and control functionality, and can be configured as discussed in Zabihian, Seyed Alireza. Toward Brain Area Sensor Wireless Network. Ecole (Canada), which Polytechnique, Montreal 2017, is hereby incorporated by reference for all purposes as if set forth herein in its entirety. Existing nanoprobe technology reduces the effect of macrophages that would otherwise respond to the large deep brain stimulation probes, thus improving the connectivity of the probe to the surrounding neurons. By detecting and modulating the unusual impedance characteristics of the catecholaminergic neuron group neurons, two-way brain computer interface functionality can be implemented, wherein signals are generated and the response to those signals can be immediately detected in other probes, to enable the interaction of the probes with the brain to be highly dimensional.

For example, consider a system that includes a remote robotic device with optical and heat sensors. The robotic device can generate hundreds of variables that define the location of the device, the types of adjacent objects, the temperatures of the adjacent objects and so forth. That data can be encoded and transmitted to implanted probes in a catecholaminergic neuron group, such as the substantia nigra pars compacta, the ventral tegmental area, the locus coeruleus or other suitable catecholaminergic neuron groups, which can use the encoded data to generate modulated signals that alert the user to the location of the robotic device, the types of adjacent objects, the temperature of the adjacent objects and so forth. The implanted probes can then be used to detect signals generated by the catecholaminergic neuron group of the user that are associated with actions to be taken by the robotic device, such as to pick up an egg, crack the egg and empty the contents of the egg into a frying pan, to monitor the temperature of the frying pan and the egg, and to remove the egg from the frying pan when it has reached the desired temperature. In this manner, a brain computer interface can be implemented that provides high dimensionality of input data to the user's neurons in manner that utilizes the unusual and nonlinear electrical properties of catecholaminergic neuron groups and that receives high dimensionality of output data from the catecholaminergic neuron groups using those unusual nonlinear electrical properties.

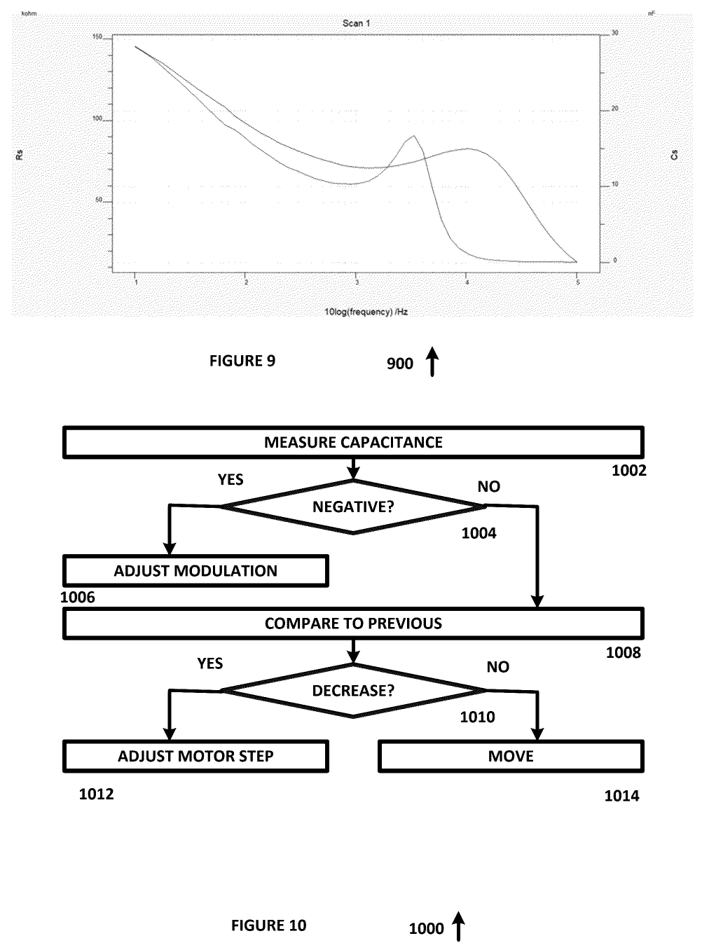

FIG. 10 is an algorithm 1000 of a test process for inserting a probe into catecholaminergic neuron tissue, in accordance with an example embodiment of the present disclosure. Algorithm 1000 can be used to insert a deep brain stimulation probe into a catecholaminergic neuron group, can be used to index allocation of nanoprobes that have been injected into a catecholaminergic neuron group, or can be used for other suitable purposes.

Algorithm 1000 begins at 1002, where electrical impedance such as resistance, capacitance and current is measured. In one example embodiment, electrical impedance can be measured by performing an electrical impedance spectroscopy test that measures capacitance and resistance at a plurality of predetermined frequencies, such as from 1 Hz to 100,000 Hz or other suitable frequency ranges. Other suitable electrical or magnetic field measurements can also or alternatively be made. The algorithm then proceeds to 1004.

At 1004, it is determined whether one or more of the measured capacitances are negative, whether current components are present at higher frequencies or if other suitable indications are present. A negative capacitance is an indication of the presence of a ferroelectric material, stored charge in an array of interacting ferritin particles or other physical processes associated with neural signaling, and negative capacitance response characteristics can be used to evaluate whether the probe location is suitable for modulation. For example, if the probe is a moveable deep brain stimulation probe, a single negative capacitance value might be a noise signal, and if capacitance values at adjacent locations or previously measured capacitance values at the same location were positive, then the decision at 1004 could be to repeat the test or to proceed to 1008. Likewise, if the probe is a stationary probe, then multiple repeated tests can be performed and can be used to determine whether the stationary probe is suitable for use in a brain computer interface. High frequency current components can also be indicative of the location of the probe in a ferritin structure that is carrying neural signals. If the decision is made to use the probe without further modification of its position or operational parameters, the algorithm proceeds to 1006, where the modulation of the probe is adjusted.

In one example embodiment, additional algorithmic processes can be used to adjust the modulation of the probe, including but not limited to 1) transmitting a signal at a predetermined frequency and measuring the impedance; 2) transmitting a signal at a predetermined frequency and measuring the response at one or more adjacent probes; 3) transmitting a signal at a predetermined frequency and receiving a report from the user; 4) transmitting an encoded signal using a predetermined encoding process and measuring the impedance; 5) transmitting an encoded signal using a predetermined encoding process and measuring the response at one or more adjacent probes; 6) transmitting an encoded signal using a predetermined encoding process and receiving a report from the user; 7) transmitting an encoded signal using an amplitude modulated encoding process and measuring the impedance; 8) transmitting an amplitude modulated encoded signal and measuring the response at one or more adjacent probes; 9) transmitting an amplitude modulated encoded signal and receiving a report from the user; 10) transmitting an encoded signal using a frequency modulated encoding process and measuring the impedance; 11) transmitting a frequency modulated encoded signal and measuring the response at one or more adjacent probes; 12) transmitting a frequency modulated encoded signal and receiving a report from the user; 13) transmitting an encoded signal using a code modulated encoding process and measuring the impedance; 14) transmitting a code modulated encoded signal and measuring the response at one or more adjacent probes; 15) transmitting a code modulated encoded signal and receiving a report from the user; 16) transmitting an encoded signal using a phase modulated encoding process and measuring the impedance; 17) transmitting a phase modulated encoded signal and measuring the response at one or more adjacent probes; 18) transmitting a phase modulated encoded signal and receiving a report from the user; 19) characterization of the ferroelectric or capacitive properties of the ferritin and neuromelanin structures that are electrically connected to the probe, or performing other suitable processes.

At 1008, the measured impedances and currents are compared to previously measured impedances. In one example embodiment, the impedance of a probe that is being inserted into a brain can be monitored to index the location of the probe, such as to determine its effective location relative to a known location or for other suitable purposes. The algorithm then proceeds to 1010.

At 1010, it is determined whether to decrease the motor step size. In one example embodiment, the probe can be inserted using a stepper motor that has a predetermined step size, such as one millimeter, 100 nanometers and so forth. If the probe is not close to a target region the step size can large, such as where the probe is being inserted into a patient's brain. When a change in impedance has been detected, the step size can be decreased to avoid overshooting the desired location. If it is determined that the motor step size should be decreased at 1010, the algorithm proceeds to 1012 where the motor step size is adjusted, such as to a smaller step size, a larger step size and so forth. The algorithm can then return to 1002 or other suitable processes can also or alternatively be implemented. Otherwise, the algorithm proceeds to 1014, where the probe is moved by an unadjusted step size, and the algorithm can then return to 1002 or other suitable processes can also or alternatively be implemented.

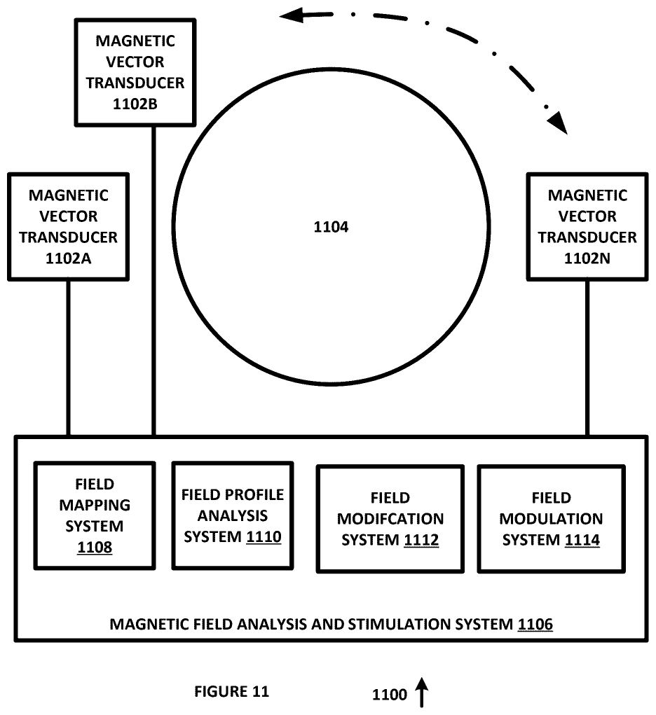

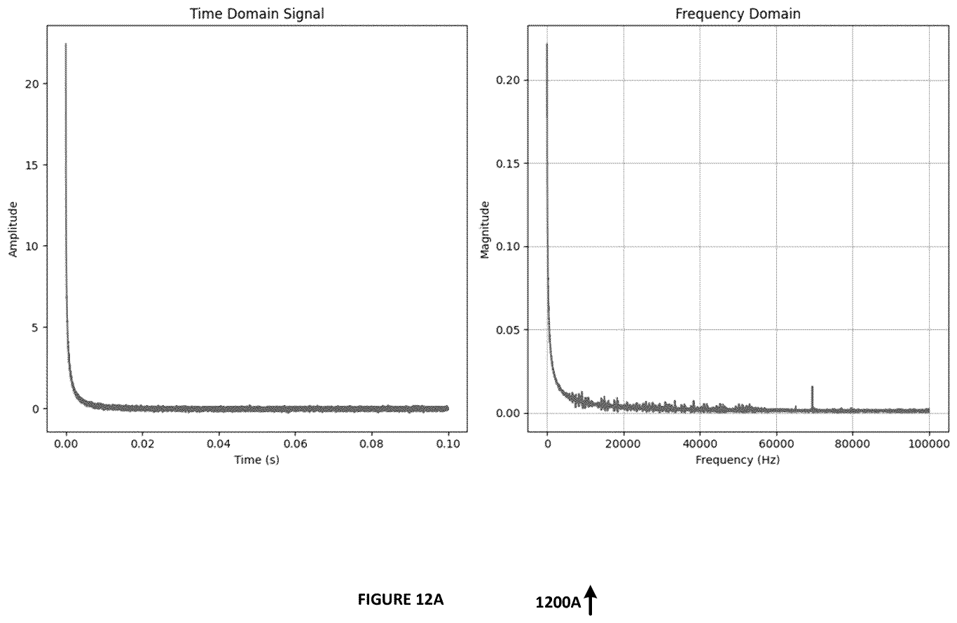

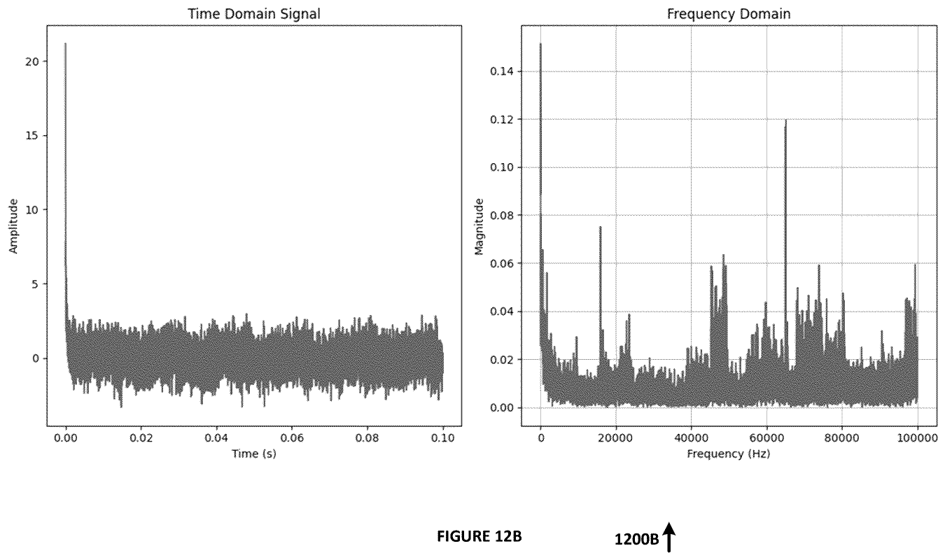

FIG. 11 is a diagram of a system 1100 for detecting and modulating ferritin-rich tissues at biologically relevant frequencies, in accordance with an example embodiment of the present disclosure. System 1100 includes magnetic vector transducers 1102A through 1102N, which can detect low magnitude magnetic field vectors at frequencies ranging from 0 to about 200 kHz or higher, and which can generate magnetic field vectors at similar frequencies. In one example embodiment, magnetic vector transducers 1102A through 1102N can be disposed at predetermined locations around the head of a user 1004, to detect magnetic field vectors as a function of time and frequency. In addition, magnetic vector transducers 1102A through 1102N can generate magnetic field vectors at controllable frequencies, magnitudes and times, as discussed further herein.

Magnetic field analysis and stimulation system 1106 is coupled to magnetic vector transducers 1102A through 1102N and is configured to detect magnetic field vectors, to analyze the magnetic field vectors, and to generate magnetic field vectors as a function of the detected magnetic fields. As discussed herein, the currents that flow within and between neural structures such as the ventral tegmental area, the substantia nigra, the red nucleus, the subthalamic nucleus, the zona incerta and other tissues containing high levels of ferritin (herein referred to ferritin tissues) can generate magnetic fields, and can respond to applied magnetic fields that are applied at the correct vectors, frequencies and magnitudes. While some response can be generated by TMS without regard to these variables, by mapping detected magnetic field vectors onto the corresponding associated neural structures, it is possible to determine whether fields are lower or higher than normal, and to stimulate those neural structures to modify the magnetic field components. Successive measurement and stimulation can modify the magnetic fields of the corresponding tissues to reduce overly excited regions and to increase under-excited regions.

Field mapping system 1108 can map detected magnetic field vectors, frequencies and magnitudes onto the associated neural structures that generate those fields. In one example embodiment, the location of magnetic vector transducers 1102A through 1102N can be mapped to the ferritin tissues that are associated with the currents that generate those magnetic fields. In this manner, the detected magnetic field vectors can be associated with the ferritin tissues to identify behavioral attributes of those tissues, such as under excitation, over excitation, excitation at frequencies that are outside of normal frequencies and so forth. For example, the substantia nigra might generate 40 to 80 KHz and 90 to 100 kHz magnetic fields at predetermined vector orientations relative to the locations of magnetic vector transducers 1102A through 1102N, and the detected magnetic field vectors can be mapped to those tissues. A similar process can be repeated for all other ferritin tissues. In this manner, a map of neural activity can be generated that is associated with the measured magnetic field vectors.

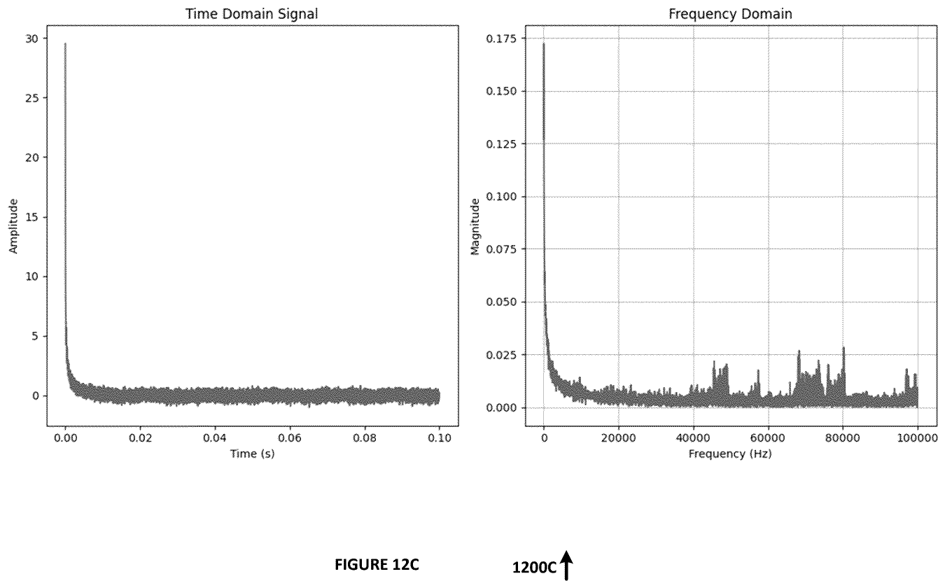

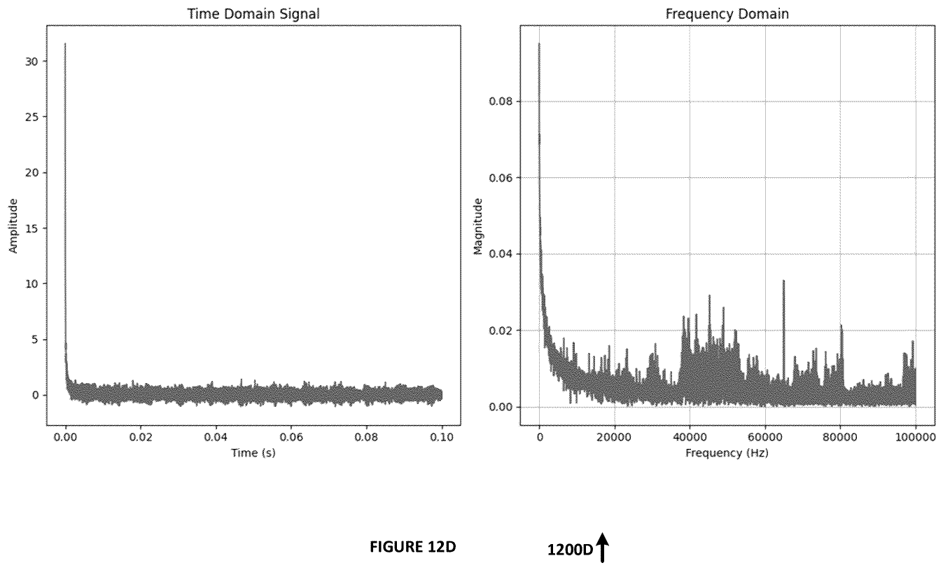

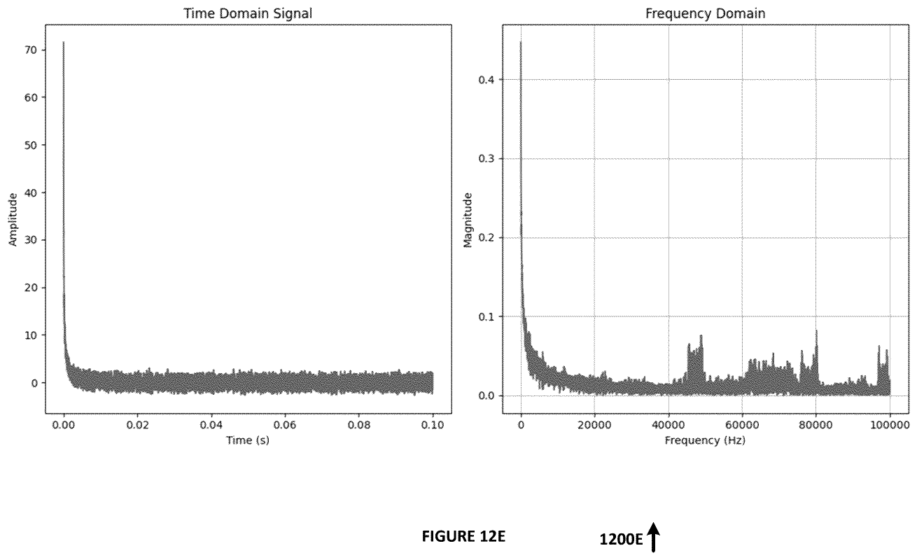

Field profile analysis system 1110 can process the map of magnetic field vectors and ferritin tissues and can identify areas where the map indicates that modulation is needed. In one example embodiment, each ferritin tissue can have a normal profile, where magnetic field vectors that exceed the normal profile can be indicative of disrupted neural processes such as anxiety, depression or other states. Because the currents that are measured in FIGS. 12A through 12E were measured using a dBS-type probe that measured currents to a common voltage, the currents are not representative of normal currents, but rather provide profiles of current magnitudes and frequencies at those ferritin tissues. Thus, the measured currents of 40 to 80 kHz and 90 to 100 kHz in the substantia nigra reflect a range of possible currents and relative magnitudes, but do not reflect normal or abnormal current levels. By making repeated measurements with different users and indexing those measurements to reported sensations from the users (such as depression, happiness, calm, anxiety and so forth), it is possible to more effectively treat conditions by modifying the magnetic fields of those tissues. In particular, the measured currents flow through ferritin structures in neurons and glial cells, such that modification of those flow paths through the proper application of external field vectors at proper frequencies and magnitudes can result in reconfiguration of the conduction states of those ferritin structures. For example, it has been shown that ferritin particles in formations similar to those in ferritin tissues can form Coulomb blockades that route currents. Applying magnetic field vectors can modify the Coulomb blockade behavior of the ferritin in the ferritin structures of ferritin tissues, which can reduce adverse sensations and increase desired sensations.

Field modification system 1112 can receive field profile analysis data and can generate field modification data. In one example embodiment, the response of ferritin tissue to external magnetic field vectors can be highly nonlinear, such that the proper magnetic field vectors, frequencies and magnitudes can result in desired magnetic field modification, but improper vectors can be ineffective or worsen user conditions. Field modification system 112 can use historical data from a database of measurements taken before and after application of magnetic field vectors, and can generate recommendation excitation parameters and predicted outcomes. Artificial intelligence models can be used to train neural networks or other suitable controls to optimize the selection of field modification vectors based on historical data in the treatment database and the measured magnetic field vectors.

Field modulation system 1114 can receive field modification data and can generate magnetic field vectors by controlling modulation frequencies, magnitudes and vectors of magnetic vector transducers 1102A through 1102N, as discussed and described further herein.

FIGS. 12A through 12E are diagrams of current waveforms measured in brain tissue using a deep brain stimulation (DBS)-type probe, in accordance with an example embodiment of the present disclosure. The current waveforms in FIGS. 12B through 12E correspond to frequency spectrums of low impedance, high frequency conducting paths associated with ferritin structures in neurons and glial cells.

Ferritin has bioelectric and biomagnetic properties that have only recently been discovered, despite extensive research into the presence and function of ferritin in tissues. FIG. 12A is a plot of the time domain and frequency domain components of currents measured in response to the voltage shown in FIG. 2 at ML −2.15 mm, AP −6.194 mm and Z 6 mm in a rat brain, but which is typical for cortical and subcortical tissues with normal levels of ferritin and neuromelanin. The frequency domain transform of the high di/dt current pulse shows some minor high frequency Gaussian noise elements, but in general the impedance at high frequencies in this tissue is very high, as expected based on prior tests. Similar frequency spectrum results were obtained at 18 other locations, and also at each of the locations for four sequential measurements (72 total measurements), such that the results were consistent and repeatable.

In contrast, FIGS. 12B through 12E show measured currents with high frequency components in the range of 18 to 22 kHz, 40 to 80 kHz ands 90 to 100 kHz in the zona incerta (FIG. 12B at ML −1.304 mm, AP −3.311 mm and Z 8.4 mm depth in the left hemisphere), 40 to 80 KHz and 90 to 100 kHz in the lateral hypothalamus (FIG. 12C at ML −2.268 mm, AP −3.965 mm and Z 8.3 mm depth in the left hemisphere), and at 40 to 80 kHz and 90 to 100 kHz in the substantia nigra (FIG. 12D ML 2.162 mm, AP −5.23 mm and Z 7.6 mm right hemisphere and FIG. 12E ML −2.621 mm, AP −5.623 mm and Z 7 mm left hemisphere). These results are typical of measurements at different adjacent depth locations (which had notable variations as a function of depth but also notable similar frequency spectrum distributions), as well as repeated measurements at the same depth location (which were mostly consistent and repeatable for each series of repeated measurements). The magnitude of the frequency bins from the Fourier transform of the time domain signal response increases with increasing frequency at approximately 40,000 Hz, although additional frequency components can be seen at lower frequencies. In general, an increase in frequency bin magnitudes of more than 20% from a previous frequency bin can be potentially significant, although the variation in the frequency bin magnitudes can complicate the analysis. Consider FIG. 12B, where the magnitude increases from less than 0.02 at approximately 30,000 to 38,000 Hz to greater than 0.4 at approximately 45,000 Hz to 50,000 Hz, with similar excursions at about 65,000 Hz to 80,000 Hz and at 95,000 Hz to 100,000 Hz. While these excursions are easy to detect from inspection, an algorithmic process for detecting the relevant frequency ranges can be more difficult to generalize. One process would be to set a first reference window to approximately 1000 Hz width, and then to compare the average magnitude of the frequency bin components in that window to the average magnitude of the frequency bin components in other 1000 Hz windows to identify bands where the magnitude is more than 50% greater or lesser, where a greater magnitude indicates that the associated band is of greater interest than the reference window, and a lesser magnitude indicates that the associated band is of lesser interest than the reference window. The size of the reference window can also be increased (e.g. to 5000 Hz) or decreased (e.g. to 100 Hz), and the process can be repeated to identify windows have the highest associated frequency bin component magnitudes (which will correspond to the most sensitive frequency ranges for subsequent stimulation and sensing).

In this manner, the frequency response of signals for a probe at a location can be automatically assessed, and used to generate test signals to determine effects on physiological variables, such as motor tremor, limb movement, heart rate respiration and so forth. Verbal report from a patient can also be used to evaluate whether stimulation at an identified frequency has a beneficial therapeutic effect. A survey of frequencies and locations and the associated therapeutic effect can thus be generated and used to improve treatment and eliminate the need for evaluation of every patient.

In another example embodiment, statistical analysis can be used to detect bands over which the frequency response is greater than the average frequency response, such as a Z score, an interquartile range, Dixon's test, Rosner's test, Grubb's test, the Mahalanobis distance or other suitable statistical metrics that can identify frequencies or frequencies bands of potential interest for therapeutic treatment. A person of skill in the art will recognize that the analysis of data such as that shown in FIGS. 12A through 12E to identify relevant features of interest (maxima, minima, ranges with the greatest variation, ranges with the least variation and so forth) is not limited to those examples discussed herein, but can include other known techniques for analysis of data.

In another example embodiment, the frequency of a signal measured at a point can be analyzed to determine the frequency bins or ranges of frequency bins that are present. Unlike the frequency interrogation process, measurement and analysis of existing signals will not identify potential therapeutic targets, but can detect signals associated with pain, anxiety, depression, schizophrenia or other disorders. Suppression of such signals might be accomplished by generating a cancellation signal in some cases, or may require stimulation of other frequencies or frequency bands. Analysis of the frequencies and frequency bins of the detected signals can be accomplished using the processes discussed above for analyzing interrogation signals.

In addition to stimulation or counter-stimulation to offset detected signals, it is possible to modulate the impedance of the probe to act as a sink to selected signals, such as to deplete the available energy for generating the signal, to cause signals to be generated at predetermined frequencies or for other suitable purposes. In one example embodiment, one or more transistors can be disposed between the electrodes of the probe and can be controllably modulated to modify the distribution of currents absorbed by the probe.

These currents will also generate magnetic fields that can be detected externally using sensitive magnetic field sensors, such as superconducting quantum interference device (SQUID) or other suitable devices. A similar process to that described above for DBS currents could also or alternatively be used for magnetic field measurement and stimulation. Existing systems and methods for transcranial magnetic stimulation (TMS) fail to account for the spatial, frequency and time-varying aspects of these currents, and thus fail to produce repeatable treatment results. The present disclosure is directed to systems and methods for using those currents for TMS, DBS and other treatments and devices. Where TMS is discussed herein, a similar process could also or alternatively be used to measure the frequency response at the probe location and then to modulate an applied signal to modify the measured signal when it exceeds normal or desired ranges. Modulation could be constant at low levels or for short durations of time at higher level, to modify Coulomb blockade configurations of ferritin in neurons and glial cells.

Several notable observations can be made from the currents shown in FIGS. 12A through 12E. First, the frequency response is similar in some regards but differs as a function of location, which is consistent with electron transport over an organically formed electron transport medium. In neural tissue, the structure of ferritin in neurons and glial cells will vary within certain parameters, such as based on the size of the neurons and glial cells, the concentration of ferritin (in glial cells) and ferritin and neuromelanin (in catecholaminergic neurons), and the connections that are formed between neurons and glial cells. While the biological processes that cause such structures to form have yet to be investigated, the presence of such structures and their ability to support electron transport have been demonstrated by these test results, as well as the potential for those structures to be modulated for treatment purposes.

Another notable observation is that the high frequency electron transport media is present in tissues with elevated ferritin levels that do not necessarily contain substantial concentrations of catecholaminergic neurons, such as the substantia nigra pars reticulata, the zona incerta and the lateral hypothalamus. While a complete survey has not been performed, it appears that tissues with endogenous levels of ferritin that are sufficient to provide magnetic resonance imaging contrast such as the red nucleus, the subthalamic nucleus and the fasciculus cerebellothalamicus, and which form a connecting structure between the hemispheres of the substantia nigra pars compacta and locus coeruleus nuclei. See, e.g. Mangia, Silvia, Shalom Michaeli, and Paul Tuite. “Magnetic resonance imaging (mri) methods in Parkinson's Disease.” Magnetic resonance imaging in movement disorders: A guide for clinicians and scientists (2013): 1-13; and Lau, Jonathan C., et al. “Direct visualization and characterization of the human zona incerta and surrounding structures.” Human brain mapping 41.16 (2020): 4500-4517. The subthalamic nucleus is a common location for deep brain stimulation, which typically uses square wave pulses to stimulate tissue based on an assumption of “volume excitation.” However, square waves have high frequency components from odd harmonic frequencies that can extend into the high frequency range, such that it is possible that the mechanism of stimulation for deep brain stimulation of the subthalamic nucleus is due to the high frequency stimulation of the ferritin electron transport structures in addition to or instead of “volume excitation.” Based on this observation, modification of a deep brain stimulation controller to provide improved stimulation by exciting frequencies associated with the electron transport region in FIGS. 12B through 12E at lower energy levels is possible. Likewise, the sequence and timing of the signals over that electron transport medium can be monitored and used to reproduce desired neural states, such as states that reduce or eliminate movement disorders, pain, emotional disorders or for other therapeutic purposes. The signals can also used be for brain-computer interface functionality, to provide 2-way capabilities.

A third observation is that the components of the current can include both the effect of the tissue, as stimulated by the potentiostat as well as current from the neural tissue. Because the tested rats were well grounded to a common ground, the current source from state generated by the triplet electrons catecholaminergic neurons and other sources provides some portion of the current. The stimulation period shown is 100 ms, and the measured response from the neural tissue will include signals generated in response to the stimulation as well as signals from normal cellular function.

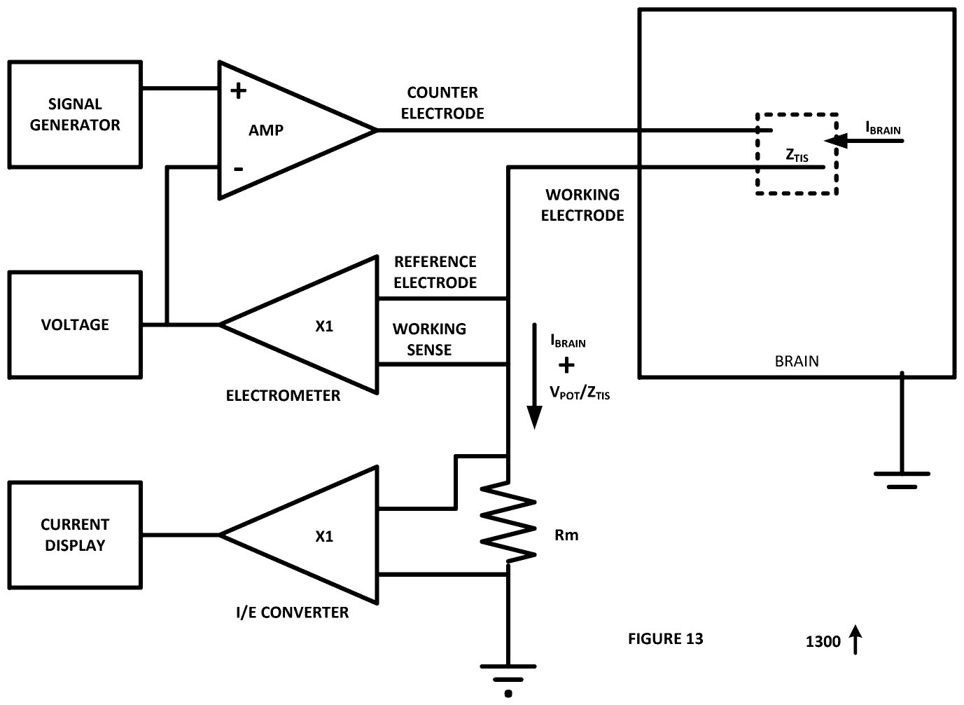

FIG. 13 is a diagram of a potentiostat system 1300 with an electrode disposed in brain tissue, in accordance with an example embodiment of the present disclosure. Potentiostat system 1300 includes a counter electrode that is held at a desired voltage and a working electrode that is used to measure a current, which is measured to common through resistance Rₘ. As shown, the tissue impedance Z_{TIS} will generate a current component equivalent to the potentiostat voltage V_{POT} divided by Z_{TIS}, but an additional current component from the brain I_{BRAIN} will also be present. In cortical tissue, the contribution from I_{BRAIN} is small, but in tissues with ferritin electron transport medium structures, the I_{BRAIN} can be more substantial and also provides an indication of the frequencies that can be monitored and stimulated for therapeutic purposes, for providing brain-computer interface functionality or for other suitable purposes.

As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

As used herein, “hardware” can include a combination of discrete components, an integrated circuit, an application-specific integrated circuit, a field programmable gate array, or other suitable hardware. As used herein, “software” can include one or more objects, agents, threads, lines of code, subroutines, separate software applications, two or more lines of code or other suitable software structures operating in two or more software applications, on one or more processors (where a processor includes one or more microcomputers or other suitable data processing units, memory devices, input-output devices, displays, data input devices such as a keyboard or a mouse, peripherals such as printers and speakers, associated drivers, control cards, power sources, network devices, docking station devices, or other suitable devices operating under control of software systems in conjunction with the processor or other devices), or other suitable software structures. In one exemplary embodiment, software can include one or more lines of code or other suitable software structures operating in a general purpose software application, such as an operating system, and one or more lines of code or other suitable software structures operating in a specific purpose software application. As used herein, the term “couple” and its cognate terms, such as “couples” and “coupled,” can include a physical connection (such as a copper conductor), a virtual connection (such as through randomly assigned memory locations of a data memory device), a logical connection (such as through logical gates of a semiconducting device), other suitable connections, or a suitable combination of such connections. The term “data” can refer to a suitable structure for using, conveying or storing data, such as a data field, a data buffer, a data message having the data value and sender/receiver address data, a control message having the data value and one or more operators that cause the receiving system or component to perform a function using the data, or other suitable hardware or software components for the electronic processing of data.

In general, a software system is a system that operates on a processor to perform predetermined functions in response to predetermined data fields. A software system is typically created as an algorithmic source code by a human programmer, and the source code algorithm is then compiled into a machine language algorithm with the source code algorithm functions, and linked to the specific input/output devices, dynamic link libraries and other specific hardware and software components of a processor, which converts the processor from a general purpose processor into a specific purpose processor. This well-known process for implementing an algorithm using a processor should require no explanation for one of even rudimentary skill in the art. For example, a system can be defined by the function it performs and the data fields that it performs the function on. As used herein, a NAME system, where NAME is typically the name of the general function that is performed by the system, refers to a software system that is configured to operate on a processor and to perform the disclosed function on the disclosed data fields. A system can receive one or more data inputs, such as data fields, user-entered data, control data in response to a user prompt or other suitable data, and can determine an action to take based on an algorithm, such as to proceed to a next algorithmic step if data is received, to repeat a prompt if data is not received, to perform a mathematical operation on two data fields, to sort or display data fields or to perform other suitable well-known algorithmic functions. Unless a specific algorithm is disclosed, then any suitable algorithm that would be known to one of skill in the art for performing the function using the associated data fields is contemplated as falling within the scope of the disclosure. For example, a message system that generates a message that includes a sender address field, a recipient address field and a message field would encompass software operating on a processor that can obtain the sender address field, recipient address field and message field from a suitable system or device of the processor, such as a buffer device or buffer system, can assemble the sender address field, recipient address field and message field into a suitable electronic message format (such as an electronic mail message, a TCP/IP message or any other suitable message format that has a sender address field, a recipient address field and message field), and can transmit the electronic message using electronic messaging systems and devices of the processor over a communications medium, such as a network. One of ordinary skill in the art would be able to provide the specific coding for a specific application based on the foregoing disclosure, which is intended to set forth exemplary embodiments of the present disclosure, and not to provide a tutorial for someone having less than ordinary skill in the art, such as someone who is unfamiliar with programming or processors in a suitable programming language. A specific algorithm for performing a function can be provided in a flow chart form or in other suitable formats, where the data fields and associated functions can be set forth in an exemplary order of operations, where the order can be rearranged as suitable and is not intended to be limiting unless explicitly stated to be limiting.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

What is claimed is:

1. A system for characterizing high frequency response of neural tissue, comprising:
a sensor configured to generate a sensed signal from a brain;
a frequency analyzer configured to receive the sensed signal and to perform a frequency analysis of the sensed signal; and
a stimulation device configured to identify a frequency component from the frequency analysis and to generate a stimulation signal at the frequency component.

2. The system of claim 1 wherein the sensor comprises a deep brain stimulation probe.

3. The system of claim 2 further comprising a current sensor coupled to the deep brain stimulation probe and configured to generate the sensed signal.

4. The system of claim 3 wherein the frequency analyzer is coupled to the current sensor and is configured to process the sensed signal to identify a plurality of frequencies in the sensed signal.

5. The system of claim 1 further comprising a current pulse generator coupled to a deep brain stimulation probe and configured to generate a high di/dt current pulse between two leads of the deep brain stimulation probe.

6. The system of claim 5 wherein the frequency analyzer is coupled to the current pulse generator and is configured to identify a plurality of frequency bands in the high di/dt pulse.

7. The system of claim 1 wherein the frequency analyzer identifies a plurality of frequencies by comparing a first portion of a frequency spectrum of the sensed signal to a second portion of the frequency spectrum of the sensed signal.

8. The system of claim 1 wherein the frequency analyzer identifies a plurality of frequencies by performing a statistical analysis of a frequency spectrum of the sensed signal.

9. The system of claim 1 wherein the sensor is a magnetic field sensor.

10. The system of claim 1 wherein the stimulation device is a magnetic field generator.

11. A method for characterizing high frequency response of neural tissue, comprising:
generating a sensed signal from a brain using a sensor;
receiving the sensed signal at a frequency analyzer and to performing a frequency analysis of the sensed signal;
identifying a frequency component from the frequency analysis; and
generating a stimulation signal at the frequency component using a stimulation device.

12. The method of claim 11 wherein the sensor comprises a deep brain stimulation probe.

13. The method of claim 12 further comprising generating the sensed signal using a current sensor coupled to the deep brain stimulation probe.

14. The method of claim 13 further comprising processing the sensed signal to identify a plurality of frequencies in the sensed signal.

15. The method of claim 11 further comprising generating a high di/dt current pulse between two leads of a deep brain stimulation probe.

16. The method of claim 15 further comprising identifying a plurality of frequency bands in the high di/dt pulse.

17. The method of claim 11 further comprising identifying a plurality of frequencies by comparing a first portion of a frequency spectrum of the sensed signal to a second portion of the frequency spectrum of the sensed signal.

18. The method of claim 11 further comprising identifying a plurality of frequencies by performing a statistical analysis of a frequency spectrum of the sensed signal.

19. The method of claim 11 wherein the sensor is a magnetic field sensor.

20. The method of claim 11 wherein the stimulation device is a magnetic field generator.
